SEQUENCE LISTING (8 sequences)

SEQ ID NO: 1 - protein, 491 aa, Homo sapiens
MELSVLLFLALLTGLLLLLVQRHPNTHDRLPPGPRPLPLLGNLLQMDRRGLLKSFLRFREKYGDVFTVHLGPRPVVMLCGVEAIREALVDKAEAFSGRGKIAMVDPFFRGYGVVFANGNRWKVLRRFSVTTMRDFGMGKRSVEERIQEEAQCLIEELRKSKGALMDPTFLFQSITANIICSIVFGKRFHYQDQEFLKMMNLFYQTFSLISSVFGQLFELFSGFLKYFPGAHRQVYKNLQEINAYIGHSVEKHRETLDPSAPKDLIDTYLLHMEKEKSNAHSEFSHQNLNLNTLSLFFAGTETTSTTLRYGFLLMLKYPHVAERVYREIEQVIGPHRPPELHDRAKMPYTEAVIYEIQRFSDLLPMGVPHIVTQHTSFRGYIIPKDTEVFLILSTALHDPHYFEKPDAFNPDHFLDANGALKKTEAFIPFSLGKRICLGEGIARAELFLFFTTILQNFSMASPVAPEDIDLTPQECGWGKIPPTYQIRFLPR

SEQ ID NO: 2 - protein, 621 aa, Homo sapiens
IQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 3 - protein, 491 aa, Homo sapiens
MELSVLLFLALLTGLLLLLVQRHPNTHDRLPPGPRPLPLLGNLLQMDRRGLLKSFLRFREKYGDVFTVHLGPRPVVMLCGVEAIREALVDKAEAFSGRGKIAMVDPFFRGYGVIFANGNRWKVLRRFSVTTMRDFGMGKRSVEERIQEEAQCLIEELRKSKGALMDPTFLFQSITANIICSIVFGKRFHYQDQEFLKMLNLFYQTFSLISSVFGQLFELFSGFLKYFPGAHRQVYKNLQEINAYIGHSVEKHRETLDPSAPKDLIDTYLLHMEKEKSNAHSEFSHQNLNLNTLSLFFAGTETTSTTLRYGFLLMLKYPHVAERVYREIEQVIGPHRPPELHDRAKMPYTEAVIYEIQRFSDLLPMGVPHIVTQHTSFRGYIIPKDTEVFLILSTALHDPHYFEKPDAFNPDHFLDANGALKKTEAFIPFSLGKRICLGEGIARAELFLFFTTILQNFSMASPVAPEDIDLTPQECGVGKIPPTYQIRFLPR

SEQ ID NO: 4 - protein, 1464 aa, Homo sapiens
MELSVLLFLALLTGLLLLLVQRHPNTHDRLPPGPRPLPLLGNLLQMDRRGLLKSFLRFREKYGDVFTVHLGPRPVVMLCGVEAIREALVDKAEAFSGRGKIAMVDPFFRGYGVVFANGNRWKVLRRFSVTTMRDFGMGKRSVEERIQEEAQCLIEELRKSKGALMDPTFLFQSITANIICSIVFGKRFHYQDQEFLKMMNLFYQTFSLISSVFGQLFELFSGFLKYFPGAHRQVYKNLQEINAYIGHSVEKHRETLDPSAPKDLIDTYLLHMEKEKSNAHSEFSHQNLNLNTLSLFFAGTETTSTTLRYGFLLMLKYPHVAERVYREIEQVIGPHRPPELHDRAKMPYTEAVIYEIQRFSDLLPMGVPHIVTQHTSFRGYIIPKDTEVFLILSTALHDPHYFEKPDAFNPDHFLDANGALKKTEAFIPFSLGKRICLGEGIARAELFLFFTTILQNFSMASPVAPEDIDLTPQECGWGKIPPTYQIRFLPRSSSTIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWSRAEGRGSLLTCGDVEENPGPMLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 5 - DNA, 987 bp, Homo sapiens
atgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggagctggaaccctga

SEQ ID NO: 6 - DNA, 60 bp, Homo sapiens
agagccgagggcaggggaagtcttctaacatgcggggacgtggaggaaaatcccgggccc

SEQ ID NO: 7 - DNA, 1047 bp, Homo sapiens
agagccgagggcaggggaagtcttctaacatgcggggacgtggaggaaaatcccgggcccatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggagctggaaccctga

SEQ ID NO: 8 - DNA, 4395 bp, Homo sapiens
atggaactcagcgtcctcctcttccttgcactcctcacaggactcttgctactcctggttcagcgccaccctaacacccatgaccgcctcccaccagggccccgccctctgccccttttgggaaaccttctgcagatggatagaagaggcctactcaaatcctttctgaggttccgagagaaatatggggacgtcttcacggtacacctgggaccgaggcccgtggtcatgctgtgtggagtagaggccatacgggaggcccttgtggacaaggctgaggccttctctggccggggaaaaatcgccatggtcgacccattcttccggggatatggtgtggtctttgccaatggaaaccgctggaaggtgcttcggcgattctctgtgaccactatgagggacttcgggatgggaaagcggagtgtggaggagcggattcaggaggaggctcagtgtctgatagaggagcttcggaaatccaagggggccctcatggaccccaccttcctcttccagtccattaccgccaacatcatctgctccatcgtctttggaaaacgattccactaccaagatcaagagttcctgaagatgatgaacttgttctaccagactttttcactcatcagctctgtattcggccagctgtttgagctcttctctggcttcttgaaatactttcctggggcacacaggcaagtttacaaaaacctgcaggaaatcaatgcttacattggccacagtgtggagaagcaccgtgaaaccctggaccccagcgcccccaaggacctcatcgacacctacctgctccacatggaaaaagagaaatccaacgcacacagtgaattcagccaccagaacctcaacctcaacacgctctcgctcttctttgctggcactgagaccaccagcaccactctccgctacggcttcctgctcatgctcaaataccctcatgttgcagagagagtctacagggagattgaacaggtgattggcccacatcgccctccagagcttcatgaccgagccaaaatgccatacacagaggcagtcatctatgagattcagagattttccgaccttctccccatgggtgtgccccacattgtcacccaacacaccagcttccgagggtacatcatccccaaggacacagaagtatttctcatcctgagcactgctctccatgacccacactactttgaaaaaccagacgccttcaatcctgaccactttctggatgccaatggggcactgaaaaagactgaagcttttatccccttctccttagggaagcggatttgtcttggtgaaggcatcgcccgtgcggaattgttcctcttcttcaccaccatcctccagaacttctccatggccagccccgtggccccagaagacatcgatctgacaccccaggagtgtggttggggcaaaatacccccaacataccagatccgcttcctgccccgctcgagctctacgattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggccggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagatggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggccggccgcatcaacaagggcgtggccaccaactggctgcgggccaaggagcctgccggggagaacggcggccgtgcgctggtgcccatgttcgtgcgcaagtcccagttccgcctgcccttcaaggccaccacgcctgtcatcatggtgggccccggcaccggggtggcacccttcataggcttcatccaggagcgggcctggctgcgacagcagggcaaggaggtgggggagacgctgctgtactacggctgccgccgctcggatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagcagagccgagggcaggggaagtcttctaacatgcggggacgtggaggaaaatcccgggcccatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggagctggaaccctga

(19) United States
(12) Patent Application Publication
(10) Pub. No.: US 2025/0320461 A1
(43) Pub. Date: Oct. 16, 2025

(54) GENETICALLY MODIFIED HUMAN STEM CELL EXPRESSING A MUTANT HUMAN CYTOCHROME P450 2B6 PROTEIN AND ITS USE THEREOF IN THE TREATMENT OF CANCER

(71) Applicants: ASSISTANCE PUBLIQUE-HOPITAUX DE PARIS, Paris (FR); UNIVERSITE DE PARIS, Paris (FR); INSTITUT NATIONAL DE LA SANTE ET DE LA RECHERCHE MEDICARE, Paris (FR); SORBONNE UNIVERSITE, Pris (FR)

(72) Inventors: Isabelle DE WAZIERS, PARIS (FR); Philippe BEAUNE, PARIS (FR); Marc SAPOVAL, ARCUEIL (FR); Olivier PELLERIN, PARIS (FR); Benjamin NAYAGOM, SAONE (FR)

(21) Appl. No.: 17/768,002

(22) PCT Filed: Oct. 13, 2020
(86) PCT No.: PCT/EP2020/078798

§ 371 Date: Apr. 11, 2022

(30) Foreign Application Priority Data
Oct. 14, 2019 (FR) FR1911385

(51) Int. Cl. C12N 5/0775; A61K 35/28; A61K 35/545; A61K 38/44; A61K 45/06; A61P 35/00; C12N 5/074; C12N 9/02

(52) U.S. Cl. CPC C12N 5/0668; A61K 35/28; A61K 35/545; A61K 38/44; A61K 45/06; A61P 35/00; C12N 5/0696; C12N 9/0042; C12N 9/0071; C12Y 106/02004; C12Y 114/14001

(57) ABSTRACT

The present invention relates to a genetically modified human stem cell, wherein said human stem cell comprises an exogenous nucleic acid comprising a region encoding a fusion protein comprising a mutant human cytochrome P450 2B6 protein (CYP2B6*) of SEQ ID No. 1, or a variant or fragment thereof and a NADPH-cytochrome P450 reductase protein of SEQ ID No. 2 or a variant or fragment thereof, operably linked to a promoter, said exogenous nucleic acid having been inserted into chromosome 17 of said human stem cell. The invention also relates to the use of said cell in the prevention and/or treatment of cancer and/or associated metastases, notably solid tumours, in particular hepatocellular carcinomas, and/or recurrent cancer and/or associated metastases.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Nov. 20, 2023, is named 17768002_ST25.txt and is 35,873 bytes in size.

### TECHNICAL FIELD OF THE INVENTION

This invention relates to a genetically modified human stem cell, wherein said human stem cell comprises an exogenous nucleic acid comprising a region encoding a fusion protein comprising a mutant human cytochrome P450 2B6 protein (CYP2B6*) of SEQ ID No. 1, or a variant or fragment thereof and a NADPH-cytochrome P450 reductase protein of SEQ ID No. 2 or a variant or fragment, operably linked to a promoter, said exogenous nucleic acid having been integrated into chromosome 17 of said human stem cell. The invention also relates to the use of this cell for the treatment of cancer, in particular solid tumours, and especially hepatocellular carcinomas.

### BACKGROUND

Around the globe, liver cancers are represented by more 841,080 new cases per year, of which 90% are hepatocellular carcinomas. Hepatocellular carcinoma is the third leading cause of cancer deaths worldwide. Chronic infection with respect to hepatitis C virus (HCV) and hepatitis B virus (HBV) or even alcoholic cirrhosis are the leading causes of hepatocellular carcinoma. Furthermore, in the last past 10 years, the emergence of non-alcoholic steatohepatitis (NASH) related to increased obesity, pre-diabetic conditions and type 2 diabetes, is an emerging cause of hepatocellular carcinoma. The treatment of hepatocellular carcinoma is essential as it can be fatal. Given the rise in patients with non-alcoholic steatohepatitis, it is highly likely that the number of patients with hepatocellular carcinoma will increase in the coming years. The spontaneous survival of patients with hepatocellular carcinoma does not exceed 15 months. BCLC staging system (Barcelona Cancer Liver Centre) distinguishes 5 types of a clinical situation.

Stages 0 and A (early) (15% of patients) define patients with a disease which has been early diagnosed, who are in good general health and who have a hepatocellular carcinoma tumour smaller than 3 cm. These patients are eligible for the only curative treatments which are tumour resection surgery and liver transplantation. The 5-year survival rate is 60%.

Stage B (intermediate) concerns patients whose general condition is stable but who have one or more hepatocellular carcinoma(s) whose size exceeds 3 cm, with no portal trunk involvement or metastases. These patients may benefit from liver chemoembolization with doxorubicin. The median survival of these patients is 20 months.

Stage C (advanced) defines a patient with hepatocellular carcinoma with portal involvement, nodal and pulmonary metastases associated with moderate impairment of general health.

The standard treatment for the advanced stage is Sorafenib, an anti-angiogenic multikinase inhibitor administered by oral route, marketed under the name Nexavar® by Onyx/Bayer. In these patients, the median survival is 11 months. Other second line treatments have recently been approved. These treatments are Regorafenib (kinase inhibitor) marketed under the name Stivarga® by Bayer and Nivolumab (anti-PD1 antibody) marketed under the name Opdivo®, by BMS. All these treatments have limited efficacy and involve major adverse events (diarrhea, weight loss, hand-foot syndrome and hypophosphatemia, high blood pressure) often caused by poor treatment compliance which leads to suboptimal outcomes.

Stage D concerns patients who have reached the palliative stage and for whom only supportive care can be offered.

Thus, there is a need for new effective forms of cancer therapy, including for solid tumours and especially hepatocellular carcinomas, in particular treatments that may provide a controlled, sustained therapy, offered alone or in combination with other therapies, whilst improving patient quality of life and reducing side effects related to treatment.

To prevent and/or treat cancer and/or associated metastases, and in particular solid tumours, notably hepatocellular carcinoma and/or associated metastases, the inventors have developed a new genetically engineered human stem cell able to metabolize cyclophosphamide and to induce the immunological death of tumour cells, by causing a strong anti-tumour immune response.

### SUMMARY OF THE INVENTION

Thus, the present invention relates to a genetically modified human stem cell, wherein said human stem cell comprises an exogenous nucleic acid comprising a region encoding a fusion protein comprising a mutant human cytochrome P450 2B6 protein (CYP2B6*) of SEQ ID No. 1, or a variant or fragment thereof and a NADPH-cytochrome P450 reductase protein of SEQ ID No. 2 or a variant or fragment thereof, operably linked to a promoter, said exogenous nucleic acid having been inserted into the intron located between exon 3 and exon 4 of the ZZEF1 gene at site 4115336 on chromosome 17 of said human stem cell, and characterized in that said genetically modified human stem cell is not an embryonic human stem cell.

Advantageously, the human stem cell is chosen from among the mesenchymal stem cells (hMSC), induced pluripotent stem cells (iPSC) and induced mesenchymal stem cells (iMSC). Advantageously, the exogenous nucleic acid is inserted into the ZZEF1 gene. Advantageously, the exogenous nucleic acid is inserted into the intron located between exon 3 and exon 4 of the ZZEF1 gene. Advantageously, the promoter used is a constitutive promoter, preferably EF1-α promoter. Advantageously, the exogenous nucleic acid also comprises a selection marker gene.

The present invention also concerns a method to obtain the genetically modified human stem cell, said human stem cell was obtained by retroviral transduction with a viral vector comprising the exogenous nucleic acid.

The present invention also concerns the genetically modified human stem cell for its use as a medicinal product and its formulation in a form suitable for trans-arterial administration.

The present invention also concerns a method for screening genetically modified human stem cells comprising the following steps:
a) transduction of the human stem cells with a viral vector comprising the exogenous nucleic acid,
b) culturing of human stem cells in a predefined culture medium,
c) screening of the genetically modified human stem cells expressing the selection marker gene on their membrane surface.

The present invention further concerns a pharmaceutical composition comprising the genetically modified human stem cell as the active substance and at least one pharmaceutically-acceptable excipient and optionally at least one second active substance, in particular an anti-cancer agent, said pharmaceutical composition being in a form suitable for trans-arterial administration.

The invention also concerns a genetically modified human stem cell or a pharmaceutical composition for its use in the prevention and/or treatment of cancer and/or associated metastases, preferably solid tumours, preferably hepatocellular carcinomas and/or associated metastases.

### DETAILED DESCRIPTION OF THE INVENTION

The present invention relates to a genetically modified human stem cell, wherein said human stem cell comprises an exogenous nucleic acid comprising a region encoding a fusion protein comprising a mutant human cytochrome P450 2B6 protein (CYP2B6*) of SEQ ID No. 1 or a variant or fragment thereof, a NADPH-cytochrome P450 reductase protein of SEQ ID No. 2 or a variant or fragment thereof, operably linked to a promoter, said exogenous nucleic acid having been integrated into chromosome 17 of said human stem cell, said genetically modified human stem cell is not an embryonic human stem cell.

Surprisingly, the inventors have shown that the integration of the exogenous nucleic acid into chromosome 17 is essential in order that the genetically modified human stem cell be able to metabolize cyclophosphamide (CPA) and induce the immunological death of the tumour cells. Additionally, this integration of exogenous nucleic acid into chromosome 17 allows the genetically modified human stem cell to maintain its morphology as well as its doubling time. Furthermore, the inventors have showed that only one copy of the exogenous nucleic acid is integrated into chromosome 17. The inventors also showed that the use of a genetically modified human stem cell according to the invention provides complete eradication of solid tumours as well as protection by a vaccine effect against recurrences and metastases.

In one particular embodiment of the invention, the human stem cell is chosen from among mesenchymal stem cells (hMSC), induced pluripotent stem cells (iPSC), and induced mesenchymal stem cells (iMSC).

As defined in the current invention, the term “mesenchymal stem cells” or “hMSC” or “human mesenchymal stem cells” refers to multi-potent stem cells capable of differentiating themselves notably into osteoblasts, chondrocytes, myocytes and adipocytes. The mesenchymal stem cells are found in the mesenchyme, the part of the embryonic mesoderm consisting of spindle-shaped cells, or star-shaped cells, not intertwined or packed loosely. As used herein, mesenchymal stem cells include, without limitation, the CD34-negative stem cells. In one embodiment of the invention, human mesenchymal stem cells were isolated from fat tissue and/or bone marrow.

As defined in the current invention, the term “induced pluripotent stem cells” or “iPSC” or “human induced pluripotent stem cells” refers to a pluripotent stem cell similar to an embryonic stem cell but which is created when somatic cells (e.g. adult cells) are reprogrammed to enter in a state similar to that of embryonic stem cell whilst maintaining the expression of important factors for maintaining the “pluripotency” of embryonic stem cells (also called CSE or PSC), i.e. their ability to be able to engage in different pathways of differentiation. Such factors may include certain embryonic genes (such as OCT4, SOX2 and LF4 transgenes). As used herein, the term “pluripotent” means a cell or cell line capable of differentiating into any differentiated cell types, e.g. the ability to develop into the three germinal layers of the body's development, in particular the endoderm, the mesoderm and the ectoderm.

As defined in the current invention, the term “induced mesenchymal stem cells” or “iMSC” or “induced human mesenchymal stem cells”, refers to mesenchymal stem cells derived from induced pluripotent stem cells (iPSC).

In one particular embodiment of the invention, the human stem cell is a mesenchymal stem cell (hMSC). In another particular embodiment of the invention, the human stem cell is an induced pluripotent stem cell (iPSC). In another particular embodiment of the invention, the human stem cell is an induced mesenchymal stem cell (iMSC).

As defined in the current invention, the term “exogenous nucleic acid” refers to a nucleic acid that is not naturally present in the human stem cell according to the invention. The exogenous nucleic acid may be a genomic DNA, a cDNA, natural DNA or DNA fully or partially obtained through chemical synthesis. According to one embodiment of the invention, the exogenous nucleic acid is of therapeutic benefit.

In a particularly advantageous embodiment of the invention, the exogenous nucleic acid comprises a region encoding a fusion protein comprising a mutant human cytochrome P450 2B6 protein (CYP2B6*) of SEQ ID No. 1 or a variant or fragment thereof, and a NADPH-cytochrome P450 reductase protein of SEQ ID No. 2 or a variant or fragment thereof.

In a particularly advantageous embodiment, the human cytochrome P450 2B6 protein is a mutant human cytochrome P450 2B6 protein, also named CYP2B6*, of SEQ ID No. 1, or a variant or fragment thereof, wherein such variant or such fragment comprises the residues 114V, 199M and 477W as shown in the amino acid sequence SEQ ID No. 1. Advantageously, said variant or fragment retains a biological activity of a protein having the amino acid sequence SEQ ID No. 1.

SEQ ID No. 1 (CYP2B6*):
MELSVLLFLALLTGLLLLLVQRHPNTHDRLPPGPRPLPLLGNLLQMDRRG
LLKSFLRFREKYGDVFTVHLGPRPVVMLCGVEAIREALVDKAEAFSGRGK
IAMVDPFFRGYGVVFANGNRWKVLRRFSVTTMRDFGMGKRSVEERIQEEA
QCLIEELRKSKGALMDPTFLFQSITANIICSIVFGKRFHYQDQEFLKMMN
LFYQTFSLISSVFGQLFELFSGFLKYFPGAHRQVYKNLQEINAYIGHSVE
KHRETLDPSAPKDLIDTYLLHMEKEKSNAHSEFSHQNLNLNTLSLFFAGT
ETTSTTLRYGFLLMLKYPHVAERVYREIEQVIGPHRPPELHDRAKMPYTE
AVIYEIQRFSDLLPMGVPHIVTQHTSFRGYIIPKDTEVFLILSTALHDPH
YFEKPDAFNPDHFLDANGALKKTEAFIPFSLGKRICLGEGIARAELFLFF
TTILQNFSMASPVAPEDIDLTPQECGWGKIPPTYQIRFLPR

The amino acid sequence of the wild-type human cytochrome P450 2B6 protein, (CYP2B6 WT) is represented by the sequence SEQ ID No. 3.

SEQ ID No. 3 (CYP2B6 WT):
MELSVLLFLALLTGLLLLLVQRHPNTHDRLPPGPRPLPLLGNLLQMDRRG
LLKSFLRFREKYGDVFTVHLGPRPVVMLCGVEAIREALVDKAEAFSGRGK
IAMVDPFFRGYGVIFANGNRWKVLRRFSVTTMRDFGMGKRSVEERIQEEA
QCLIEELRKSKGALMDPTFLFQSITANIICSIVFGKRFHYQDQEFLKMLN
LFYQTFSLISSVFGQLFELFSGFLKYFPGAHRQVYKNLQEINAYIGHSVE
KHRETLDPSAPKDLIDTYLLHMEKEKSNAHSEFSHQNLNLNTLSLFFAGT
ETTSTTLRYGFLLMLKYPHVAERVYREIEQVIGPHRPPELHDRAKMPYTE
AVIYEIQRFSDLLPMGVPHIVTQHTSFRGYIIPKDTEVFLILSTALHDPH
YFEKPDAFNPDHFLDANGALKKTEAFIPFSLGKRICLGEGIARAELFLFF
TTILQNFSMASPVAPEDIDLTPQECGVGKIPPTYQIRFLPR

Advantageously, the mutant human cytochrome P450 2B6 protein (CYP2B6*) has an affinity for CPA of more than 8 times greater than that of the wild-type protein (CYP2B6 WT), while retaining the same Vmax, due, notably, to the substitutions I114V, L199M and V477W, as shown in SEQ ID No. 1. As described below, variants and fragments of the amino acid sequence SEQ ID No. 1 are encompassed within the scope according to the invention. However, all mutant human cytochrome P450 2B6 protein, variants and fragments according to the invention as described here retain Val at the position corresponding to residue 114 of the amino acid sequence SEQ ID No. 1, Met at the position corresponding to residue 199 of the amino acid sequence SEQ ID No. 1 and Trp at the position corresponding to residue 477 of the amino acid sequence SEQ ID No. 1.

The amino acid sequence of wild-type NADPH-cytochrome P450 reductase protein (NADPH WT), is represented by the sequence SEQ ID No. 2. Variants and fragments of the NADPH-cytochrome P450 reductase protein of amino acid sequence SEQ ID No. 2, described below, are also encompassed within the scope according to the invention.

SEQ ID No. 2 (NADPH WT):
IQTLTSSVRESSFVEKMKKTGRNIIVFYGSQTGTAEEFANRLSKDAHRYG
MRGMSADPEEYDLADLSSLPEIDNALVVFCMATYGEGDPTDNAQDFYDWL
QETDVDLSGVKFAVFGLGNKTYEHFNAMGKYVDKRLEQLGAQRIFELGLG
DDDGNLEEDFITWREQFWPAVCEHFGVEATGEESSIRQYELVVHTDIDAA
KVYMGEMGRLKSYENQKPPFDAKNPFLAAVTTNRKLNQGTERHLMHLELD
ISDSKIRYESGDHVAVYPANDSALVNQLGKILGADLDVVMSLNNLDEESN
KKHPFPCPTSYRTALTYYLDITNPPRTNVLYELAQYASEPSEQELLRKMA
SSSGEGKELYLSWVVEARRHILAILQDCPSLRPPIDHLCELLPRLQARYY
SIASSSKVHPNSVHICAVVVEYETKAGRINKGVATNWLRAKEPAGENGGR
ALVPMFVRKSQFRLPFKATTPVIMVGPGTGVAPFIGFIQERAWLRQQGKE
VGETLLYYGCRRSDEDYLYREELAQFHRDGALTQLNVAFSREQSHKVYVQ
HLLKQDREHLWKLIEGGAHIYVCGDARNMARDVQNTFYDIVAELGAMEHA
QAVDYIKKLMTKGRYSLDVWS

Variant proteins may be naturally occurring variants, such as splice variants, alleles and isoforms, or they may be produced by recombinant means. Variations in amino acid sequence may be introduced by substitution, deletion or insertion of one or more codons into the nucleic acid sequence encoding the protein that results in a change in the amino acid sequence of the protein. Optionally the variation is by substitution of 1, 2, 3, 4, 5, 6, 7, 8, 9, 10, 11, 12, 13, 14, 15, 16, 17, 18, 19, 20 or more amino acids with any other amino acid in the protein. Additionally or alternatively, the variation may be by addition or deletion of 1, 2, 3, 4, 5, 6, 7, 8, 9, 10, 11, 12, 13, 14, 15, 16, 17, 18, 19, 20 or more amino acids within the protein. Amino acid substitutions may be conservative or non-conservative. Preferably, substitutions are conservative substitutions, in which one amino acid is substituted for another amino acid with similar structural and/or chemical properties. Exemplary conservative substitutions are listed below.
Ala (A) Val; Leu; Ile
Arg (R) Lys; Gln; Asn
Asn (N) Gln; His; Lys
Asp (D) Glu
Cys (C) Ser
Gln (Q) Asn
Glu (E) Asp
Gly (G) Pro; Ala; ala
His (H) Asn; Gln; Lys; Arg;
Ile (I) Leu; Val; Met; Ala;
norleucine Leu
Leu (L) norleucine; Ile; Met; Ala; Phe
Lys (K) Arg; Gln; Asn
Met (M) Leu; Phe; Ile
Phe (F) Leu; Val; Ile; Ala; Tyr
Pro (P) Ala
Ser (S) Thr
Thr (T) Ser
Trp (W) Tyr; Phe;
Tyr (Y) Trp; Phe; Thr; Ser;
Val (V) Ile; Leu; Met; Phe; Ala; norleucine.

Variant proteins may include proteins that have at least about 80% amino acid sequence identity with a polypeptide sequence disclosed herein. Advantageously, a variant protein will have at least about 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of amino acid sequence SEQ ID No. 1 and/or SEQ ID No. 2.

Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both. Methods for sequence alignment and determination of sequence identity are well known in the art, for example using publicly available computer software such as BioPerl, BLAST, BLAST-2, CS-BLAST, FASTA, ALIGN, ALIGN-2, LALIGN, Jaligner, matcher or Megalign (DNASTAR) software.

Fragments of the proteins and variant proteins disclosed herein are also encompassed in context of the invention. Such fragments may be truncated at the N-terminus or C-terminus, or may lack internal residues beyond the N-terminus or C-terminus, for example, when compared with a full length protein. Certain fragments lack amino acid residues that are not essential for enzymatic activity. Advantageously, said fragments are at least about 10, 20, 30, 40, 50, 60, 70, 80, 90, 100, 110, 120, 150, 250, 300, 350, 400, 450, 500 or more amino acids in length.

Preferred fragments of the proteins disclosed herein comprise all or a part of the active site. Preferred fragments of mutant human cytochrome P450 2B6 proteins (CYP2B6*) comprise or consist of amino acids 1 to 490 of the full length sequence SEQ ID No 1. Preferred fragments of NADPH-cytochrome P450 reductase protein comprise or consist of fragments comprising or consisting of amino acids 60 to 680 of the amino acids sequence SEQ ID No 2.

Variants and fragments of the invention preferably retain a biological activity of the full-length protein. Variants and fragments of human cytochrome P450 2B6 protein advantageously have an activity of oxidising a substrate such as cyclophosphamide (CPA), or other substrate, in particular by catalysing hydroxylation of 4-OH-CPA.

In one particularly advantageous embodiment, said variants and fragments have an affinity for cyclophosphamide (CPA) greater than that of human cytochrome P450 2B6 protein of sequence SEQ ID No. 3, preferably at least 2, 3, 4, 5, 6, 7, 8, 9 or 10 times that of the wild-type sequence.

In one particularly advantageous embodiment, said variants and fragments have an affinity for CPA the same as, substantially the same as, or greater than, that of the mutant human cytochrome P450 2B6 protein (CYP2B6*) of SEQ ID No 1. Methods for assaying said activity and affinity are well known of the skilled person in the art and are notably described in Nguyen et al, Mol Pharmacol 2008, 73:1122-1133.

Variants and fragments of NADPH-cytochrome P450 reductase protein preferably have the activity of reduction of cytochrome c, preferably in a NADPH-dependent fashion.

In a preferred embodiment, said variants and fragments have an activity the same as, substantially the same as, or greater than, that of the full-length NADPH-cytochrome P450 reductase protein (SEQ ID No 2). Methods for assaying said activity are well known of the skilled person in the art and are notably described in Yasukochi et al; Arch Biochem Biophys 1980, 202:491-498.

The skilled person will be able to determine amino acid residues which may be inserted, substituted or deleted without adversely affecting the activity of the protein using knowledge of the protein structure available in the art and publicly available molecular modelling techniques (see for example Nguyen et al, Mol Pharmacol 2008, 73:1122-1133). The variation allowed may be determined by systematically making insertions, deletions or substitutions of amino acids in the sequence and testing the resulting variants for activity exhibited by the wild-type protein.

As defined in the invention, the term “fusion protein” refers to a chimeric protein created by joining two or more genes encoding separate proteins or protein fragments, such as different protein domains, into a single reading frame encoding a single translated protein.

The fusion protein of the present invention preferably comprises:
a mutant human cytochrome P450 2B6 protein (CYP2B6*) of SEQ ID No. 1 or a variant or fragment thereof, and
a NADPH-cytochrome P450 reductase protein of SEQ ID No. 2 or a variant or fragment thereof, as described above.

In a particularly advantageous embodiment, said fusion protein of sequence SEQ ID No. 4 comprises:
residues 1 to 490 of the mutant human cytochrome P450 2B6 protein (CYP2B6*) of SEQ ID No. 1 or a variant or fragment thereof, and
residues 57 to 678 of the NADPH-cytochrome P450 reductase protein of SEQ ID No. 2 or a variant or fragment thereof.

SEQ ID No. 4 (fusion protein):
MELSVLLFLALLTGLLLLLVQRHPNTHDRLPPGPRPLPLLGNLLQMDRRG
LLKSFLRFREKYGDVFTVHLGPRPVVMLCGVEAIREALVDKAEAFSGRGK
IAMVDPFFRGYGVVFANGNRWKVLRRFSVTTMRDFGMGKRSVEERIQEEA
QCLIEELRKSKGALMDPTFLFQSITANIICSIVFGKRFHYQDQEFLKMMN
LFYQTFSLISSVFGQLFELFSGFLKYFPGAHRQVYKNLQEINAYIGHSVE
KHRETLDPSAPKDLIDTYLLHMEKEKSNAHSEFSHQNLNLNTLSLFFAGT
ETTSTTLRYGFLLMLKYPHVAERVYREIEQVIGPHRPPELHDRAKMPYTE
AVIYEIQRFSDLLPMGVPHIVTQHTSFRGYIIPKDTEVFLILSTALHDPH
YFEKPDAFNPDHFLDANGALKKTEAFIPFSLGKRICLGEGIARAELFLFF
TTILQNFSMASPVAPEDIDLTPQECGWGKIPPTYQIRFLPRSSSTIQTLT
SSVRESSFVEKMKKTGRNIIVFYGSQTGTAEEFANRLSKDAHRYGMRGMS
ADPEEYDLADLSSLPEIDNALVVFCMATYGEGDPTDNAQDFYDWLQETDV
DLSGVKFAVFGLGNKTYEHFNAMGKYVDKRLEQLGAQRIFELGLGDDDGN
LEEDFITWREQFWPAVCEHFGVEATGEESSIRQYELVVHTDIDAAKVYMG
EMGRLKSYENQKPPFDAKNPFLAAVTTNRKLNQGTERHLMHLELDISDSK
IRYESGDHVAVYPANDSALVNQLGKILGADLDVVMSLNNLDEESNKKHPF
PCPTSYRTALTYYLDITNPPRTNVLYELAQYASEPSEQELLRKMASSSGE
GKELYLSWVVEARRHILAILQDCPSLRPPIDHLCELLPRLQARYYSIASS
SKVHPNSVHICAVVVEYETKAGRINKGVATNWLRAKEPAGENGGRALVPM
FVRKSQFRLPFKATTPVIMVGPGTGVAPFIGFIQERAWLRQQGKEVGETL
LYYGCRRSDEDYLYREELAQFHRDGALTQLNVAFSREQSHKVYVQHLLKQ
DREHLWKLIEGGAHIYVCGDARNMARDVQNTFYDIVAELGAMEHAQAVDY
IKKLMTKGRYSLDVWSRAEGRGSLLTCGDVEENPGPMLVRRGARAGPRMP
RGWTALCLLSLLPSGFMSLDNNGTATPELPTQGTFSNVSTNVSYQETTTP
STLGSTSLHPVSQHGNEATTNITETTVKFTSTSVITSVYGNTNSSVQSQT
SVISTVFTTPANVSTPETTLKPSLSPGNVSDLSTTSTSLATSPTKPYTSS
SPILSDIKAEIKCSGIREVKLTQGICLEQNKTSSCAEFKKDRGEGLARVL
CGEEQADADAGAQVCSLLLAQSEVRPQCLLLVLANRTEISSKLQLMKKHQ
SDLKKLGILDFTEQDVASHQSYSQKTLIALVTSGALLAVLGITGYFLMNR
RSWSPTGERLELEP*.

The mutant human cytochrome P450 2B6 protein (CYP2B6*) of SEQ ID No. 1 or a variant or fragment thereof may be upstream or downstream of the NADPH-cytochrome P450 reductase protein of SEQ ID No. 2 or a variant or fragment thereof. Preferably, the mutant human cytochrome P450 2B6 protein (CYP2B6*) of SEQ ID No. 1 or variant or fragment thereof is upstream of the NADPH-cytochrome P450 reductase protein of SEQ ID No. 2, or a variant or fragment thereof.

When context permits, reference herein to “the proteins according to the invention”, “the proteins disclosed herein” should be understood to encompass said fusion proteins.

The proteins or protein fragments making up the fusion protein may be separated by a linker peptide sequence or spacer. The linker peptide sequence, also named “linker” or the spacer, serves to separate the proteins or protein fragments of the fusion protein and aid effective folding and activity of the individual components. The linker peptide sequence or the spacer may comprise an enzyme cleavage site to permit the component polypeptides to be separates by enzymatic digestion. The linker peptide sequence or the spacer may be, for example, 1, 2, 3, 4, 5, 6, 7, 8, 9, 10, 11, 12, 13, 14, 15 amino acids or more in length. Preferably, the linker peptide sequence or the spacer is less than 10, less than 9, less than 8, less than 7, less than 6 or less than 5 amino acids in length. Exemplary linkers peptide sequence or spacer for use in the fusion proteins of the present invention comprise (Ser) n Thr, where “n” is any whole integer, preferably 1, 2, 3, 4, 5, 6, 7, 8, or 9. In a preferred aspect, “n” is 5. In another preferred aspect, “n” is 3.

In one particular embodiment of the invention, the exogenous nucleic acid is not integrated in an aleatory manner in the genome of the human stem cell. In contrast, the exogenous nucleic acid is integrated into the ZZEF1 gene (Zinc finger ZZ-type and EF-hand domain containing). Advantageously, one sole copy of the exogenous nucleic acid is integrated into the ZZEF1 gene.

In one particular embodiment of the invention, the exogenous nucleic acid is integrated into the intron located between exon 3 and exon 4 of the ZZEF1 gene. Advantageously, the exogenous nucleic acid is integrated between the exon 3 and exon 4 of the ZZEF1 gene at site 4115336 on chromosome 17.

In one particular embodiment of the invention, the promoter to which is operably linked the region encoding the fusion protein comprising a mutant human cytochrome P450 2B6 protein (CYP2B6*) of SEQ ID No. 1 or a variant or fragment thereof, and a NADPH-cytochrome P450 reductase protein of SEQ ID No. 2 or a variant or fragment thereof, is a constitutive promoter. Advantageously, the constitutive promoter can be selected from the CMV promoter, the CBA promoter and the EF1-α promoter. Advantageously, the constitutive promoter is the EF1-α Promoter.

In one particular embodiment of the invention, the exogenous nucleic acid further comprises a selection marker gene. “Selection marker gene” as defined in the current invention refers to a coding sequence for a selectable marker protein, which allows the selective retention of human stem cells having integrated the exogenous nucleic acid during the culture and the propagation of cells. Advantageously, the selection marker gene is CD34 gene, advantageously the CD34 gene which has been spliced, and which is also called spliced CD34 gene.

Advantageously, the selection marker gene is spliced CD34 gene of sequence SEQ ID No. 5.

SEQ ID No. 5 (spliced CD34 selection marker gene):
ATGCTGGTCCGCAGGGGCGCGCGCGCAGGGCCCAGGATGCCGCGGGGCTG
GACCGCGCTTTGCTTGCTGAGTTTGCTGCCTTCTGGGTTCATGAGTCTTG
ACAACAACGGTACTGCTACCCCAGAGTTACCTACCCAGGGAACATTTTCA
AATGTTTCTACAAATGTATCCTACCAAGAAACTACAACACCTAGTACCCT
TGGAAGTACCAGCCTGCACCCTGTGTCTCAACATGGCAATGAGGCCACAA
CAAACATCACAGAAACGACAGTCAAATTCACATCTACCTCTGTGATAACC
TCAGTTTATGGAAACACAAACTCTTCTGTCCAGTCACAGACCTCTGTAAT
CAGCACAGTGTTCACCACCCCAGCCAACGTTTCAACTCCAGAGACAACCT
TGAAGCCTAGCCTGTCACCTGGAAATGTTTCAGACCTTTCAACCACTAGC
ACTAGCCTTGCAACATCTCCCACTAAACCCTATACATCATCTTCTCCTAT
CCTAAGTGACATCAAGGCAGAAATCAAATGTTCAGGCATCAGAGAAGTGA
AATTGACTCAGGGCATCTGCCTGGAGCAAAATAAGACCTCCAGCTGTGCG
GAGTTTAAGAAGGACAGGGGAGAGGGCCTGGCCCGAGTGCTGTGTGGGGA
GGAGCAGGCTGATGCTGATGCTGGGGCCCAGGTATGCTCCCTGCTCCTTG
CCCAGTCTGAGGTGAGGCCTCAGTGTCTACTGCTGGTCTTGGCCAACAGA
ACAGAAATTTCCAGCAAACTCCAACTTATGAAAAAGCACCAATCTGACCT
GAAAAAGCTGGGGATCCTAGATTTCACTGAGCAAGATGTTGCAAGCCACC
AGAGCTATTCCCAAAAGACCCTGATTGCACTGGTCACCTCGGGAGCCCTG
CTGGCTGTCTTGGGCATCACTGGCTATTTCCTGATGAATCGCCGCAGCTG
GAGCCCCACAGGAGAAAGGCTGGAGCTGGAACCCTGA

Use of spliced CD34 gene allows to select the human stem cells which have integrated the exogenous nucleic acid without having signal transduction activity. In a particularly advantageous embodiment, the spliced CD34 selection marker gene is operably linked to a nucleic acid of sequence SEQ ID No. 6 encoding the peptide 2A.

SEQ ID No. 6 (peptide 2A):
AGAGCCGAGGGCAGGGGAAGTCTTCTAACATGCGGGGACGTGGAGGAAAA
TCCCGGGCCC

The spliced CD34 selection marker gene is operably linked to a nucleic acid encoding the peptide 2A and is represented by the nucleic acids sequence of SEQ ID No. 7.

SEQ ID No. 7 (Spliced CD34selection marker gene-
peptide 2A):
AGAGCCGAGGGCAGGGGAAGTCTTCTAACATGCGGGGACGTGGAGGAAAA
TCCCGGGCCCATGCTGGTCCGCAGGGGCGCGCGCGCAGGGCCCAGGATGC
CGCGGGGCTGGACCGCGCTTTGCTTGCTGAGTTTGCTGCCTTCTGGGTTC
ATGAGTCTTGACAACAACGGTACTGCTACCCCAGAGTTACCTACCCAGGG
AACATTTTCAAATGTTTCTACAAATGTATCCTACCAAGAAACTACAACAC
CTAGTACCCTTGGAAGTACCAGCCTGCACCCTGTGTCTCAACATGGCAAT
GAGGCCACAACAAACATCACAGAAACGACAGTCAAATTCACATCTACCTC
TGTGATAACCTCAGTTTATGGAAACACAAACTCTTCTGTCCAGTCACAGA
CCTCTGTAATCAGCACAGTGTTCACCACCCCAGCCAACGTTTCAACTCCA
GAGACAACCTTGAAGCCTAGCCTGTCACCTGGAAATGTTTCAGACCTTTC
AACCACTAGCACTAGCCTTGCAACATCTCCCACTAAACCCTATACATCAT
CTTCTCCTATCCTAAGTGACATCAAGGCAGAAATCAAATGTTCAGGCATC
AGAGAAGTGAAATTGACTCAGGGCATCTGCCTGGAGCAAAATAAGACCTC
CAGCTGTGCGGAGTTTAAGAAGGACAGGGGAGAGGGCCTGGCCCGAGTGC
TGTGTGGGGAGGAGCAGGCTGATGCTGATGCTGGGGCCCAGGTATGCTCC
CTGCTCCTTGCCCAGTCTGAGGTGAGGCCTCAGTGTCTACTGCTGGTCTT
GGCCAACAGAACAGAAATTTCCAGCAAACTCCAACTTATGAAAAAGCACC
AATCTGACCTGAAAAAGCTGGGGATCCTAGATTTCACTGAGCAAGATGTT
GCAAGCCACCAGAGCTATTCCCAAAAGACCCTGATTGCACTGGTCACCTC
GGGAGCCCTGCTGGCTGTCTTGGGCATCACTGGCTATTTCCTGATGAATC
GCCGCAGCTGGAGCCCCACAGGAGAAAGGCTGGAGCTGGAACCCTGA

Another object of the invention is an expression vector, for example a viral vector, comprising the exogenous nucleic acid such as defined herein. In the context of the invention, the term “vector” refers to a polynucleotide sequence containing the exogenous nucleic acid as defined herein and control sequences in operable linkage, so that human stem cells transformed with these sequences are capable of producing the encoded proteins, in particular the fusion protein comprising a mutant human cytochrome P450 2B6 protein (CYP2B6*) of SEQ ID No. 1 or a variant or fragment thereof, and a NADPH-cytochrome P450 reductase protein of SEQ ID No. 2 or a variant or fragment thereof.

In one particular embodiment, the human stem cell was obtained by retroviral transduction, using a viral vector comprising the exogenous nucleic acid.

The vector usually comprises a promoter, signals for initiation and termination of translation, as well as appropriate regions for regulation of the transcription. In one particularly advantageous embodiment of the invention, the vector comprises a nucleic acid of SEQ ID No 8, comprising:
the exogenous nucleic acid comprising a region encoding a fusion protein comprising a mutant human cytochrome P450 2B6 protein (CYP2B6*) of SEQ ID No. 1 or a variant or fragment thereof, and a NADPH-cytochrome P450 reductase protein of SEQ ID No. 2 or a variant or fragment thereof as defined above, and
the selection marker gene operably linked to the nucleic acid encoding peptide 2A as defined above.

SEQ ID No 8 (vectorized nucleic acid):
ATGGAACTCAGCGTCCTCCTCTTCCTTGCACTCCTCACAGGACTCTTGCT
ACTCCTGGTTCAGCGCCACCCTAACACCCATGACCGCCTCCCACCAGGGC
CCCGCCCTCTGCCCCTTTTGGGAAACCTTCTGCAGATGGATAGAAGAGGC
CTACTCAAATCCTTTCTGAGGTTCCGAGAGAAATATGGGGACGTCTTCAC
GGTACACCTGGGACCGAGGCCCGTGGTCATGCTGTGTGGAGTAGAGGCCA
TACGGGAGGCCCTTGTGGACAAGGCTGAGGCCTTCTCTGGCCGGGGAAAA
ATCGCCATGGTCGACCCATTCTTCCGGGGATATGGTGTGGTCTTTGCCAA
TGGAAACCGCTGGAAGGTGCTTCGGCGATTCTCTGTGACCACTATGAGGG
ACTTCGGGATGGGAAAGCGGAGTGTGGAGGAGCGGATTCAGGAGGAGGCT
CAGTGTCTGATAGAGGAGCTTCGGAAATCCAAGGGGGCCCTCATGGACCC
CACCTTCCTCTTCCAGTCCATTACCGCCAACATCATCTGCTCCATCGTCT
TTGGAAAACGATTCCACTACCAAGATCAAGAGTTCCTGAAGATGATGAAC
TTGTTCTACCAGACTTTTTCACTCATCAGCTCTGTATTCGGCCAGCTGTT
TGAGCTCTTCTCTGGCTTCTTGAAATACTTTCCTGGGGCACACAGGCAAG
TTTACAAAAACCTGCAGGAAATCAATGCTTACATTGGCCACAGTGTGGAG
AAGCACCGTGAAACCCTGGACCCCAGCGCCCCCAAGGACCTCATCGACAC
CTACCTGCTCCACATGGAAAAAGAGAAATCCAACGCACACAGTGAATTCA
GCCACCAGAACCTCAACCTCAACACGCTCTCGCTCTTCTTTGCTGGCACT
GAGACCACCAGCACCACTCTCCGCTACGGCTTCCTGCTCATGCTCAAATA
CCCTCATGTTGCAGAGAGAGTCTACAGGGAGATTGAACAGGTGATTGGCC
CACATCGCCCTCCAGAGCTTCATGACCGAGCCAAAATGCCATACACAGAG
GCAGTCATCTATGAGATTCAGAGATTTTCCGACCTTCTCCCCATGGGTGT
GCCCCACATTGTCACCCAACACACCAGCTTCCGAGGGTACATCATCCCCA
AGGACACAGAAGTATTTCTCATCCTGAGCACTGCTCTCCATGACCCACAC
TACTTTGAAAAACCAGACGCCTTCAATCCTGACCACTTTCTGGATGCCAA
TGGGGCACTGAAAAAGACTGAAGCTTTTATCCCCTTCTCCTTAGGGAAGC
GGATTTGTCTTGGTGAAGGCATCGCCCGTGCGGAATTGTTCCTCTTCTTC
ACCACCATCCTCCAGAACTTCTCCATGGCCAGCCCCGTGGCCCCAGAAGA
CATCGATCTGACACCCCAGGAGTGTGGTTGGGGCAAAATACCCCCAACAT
ACCAGATCCGCTTCCTGCCCCGCTCGAGCTCTACGATTCAGACATTGACC
TCCTCTGTCAGAGAGAGCAGCTTTGTGGAAAAGATGAAGAAAACGGGGAG
GAACATCATCGTGTTCTACGGCTCCCAGACGGGGACTGCAGAGGAGTTTG
CCAACCGCCTGTCCAAGGACGCCCACCGCTACGGGATGCGAGGCATGTCA
GCGGACCCTGAGGAGTATGACCTGGCCGACCTGAGCAGCCTGCCAGAGAT
CGACAACGCCCTGGTGGTTTTCTGCATGGCCACCTACGGTGAGGGAGACC
CCACCGACAATGCCCAGGACTTCTACGACTGGCTGCAGGAGACAGACGTG
GATCTCTCTGGGGTCAAGTTCGCGGTGTTTGGTCTTGGGAACAAGACCTA
CGAGCACTTCAATGCCATGGGCAAGTACGTGGACAAGCGGCTGGAGCAGC
TCGGCGCCCAGCGCATCTTTGAGCTGGGGTTGGGCGACGACGATGGGAAC
TTGGAGGAGGACTTCATCACCTGGCGAGAGCAGTTCTGGCCGGCCGTGTG
TGAACACTTTGGGGTGGAAGCCACTGGCGAGGAGTCCAGCATTCGCCAGT
ACGAGCTTGTGGTCCACACCGACATAGATGCGGCCAAGGTGTACATGGGG
GAGATGGGCCGGCTGAAGAGCTACGAGAACCAGAAGCCCCCCTTTGATGC
CAAGAATCCGTTCCTGGCTGCAGTCACCACCAACCGGAAGCTGAACCAGG
GAACCGAGCGCCACCTCATGCACCTGGAATTGGACATCTCGGACTCCAAA
ATCAGGTATGAATCTGGGGACCACGTGGCTGTGTACCCAGCCAACGACTC
TGCTCTCGTCAACCAGCTGGGCAAAATCCTGGGTGCCGACCTGGACGTCG
TCATGTCCCTGAACAACCTGGATGAGGAGTCCAACAAGAAGCACCCATTC
CCGTGCCCTACGTCCTACCGCACGGCCCTCACCTACTACCTGGACATCAC
CAACCCGCCGCGTACCAACGTGCTGTACGAGCTGGCGCAGTACGCCTCGG
AGCCCTCGGAGCAGGAGCTGCTGCGCAAGATGGCCTCCTCCTCCGGCGAG
GGCAAGGAGCTGTACCTGAGCTGGGTGGTGGAGGCCCGGAGGCACATCCT
GGCCATCCTGCAGGACTGCCCGTCCCTGCGGCCCCCCATCGACCACCTGT
GTGAGCTGCTGCCGCGCCTGCAGGCCCGCTACTACTCCATCGCCTCATCC
TCCAAGGTCCACCCCAACTCTGTGCACATCTGTGCGGTGGTTGTGGAGTA
CGAGACCAAGGCCGGCCGCATCAACAAGGGCGTGGCCACCAACTGGCTGC
GGGCCAAGGAGCCTGCCGGGGAGAACGGCGGCCGTGCGCTGGTGCCCATG
TTCGTGCGCAAGTCCCAGTTCCGCCTGCCCTTCAAGGCCACCACGCCTGT
CATCATGGTGGGCCCCGGCACCGGGGGGCACCCTTCATAGGCTTCATCCA
GGAGCGGGCCTGGCTGCGACAGCAGGGCAAGGAGGTGGGGGAGACGCTGC
TGTACTACGGCTGCCGCCGCTCGGATGAGGACTACCTGTACCGGGAGGAG
CTGGCGCAGTTCCACAGGGACGGTGCGCTCACCCAGCTCAACGTGGCCTT
CTCCCGGGAGCAGTCCCACAAGGTCTACGTCCAGCACCTGCTAAAGCAAG
ACCGAGAGCACCTGTGGAAGTTGATCGAAGGCGGTGCCCACATCTACGTC
TGTGGGGATGCACGGAACATGGCCAGGGATGTGCAGAACACCTTCTACGA
CATCGTGGCTGAGCTCGGGGCCATGGAGCACGCGCAGGCGGTGGACTACA
TCAAGAAACTGATGACCAAGGGCCGCTACTCCCTGGACGTGTGGAGCAGA
GCCGAGGGCAGGGGAAGTCTTCTAACATGCGGGGACGTGGAGGAAAATCC
CGGGCCCATGCTGGTCCGCAGGGGCGCGCGCGCAGGGCCCAGGATGCCGC
GGGGCTGGACCGCGCTTTGCTTGCTGAGTTTGCTGCCTTCTGGGTTCATG
AGTCTTGACAACAACGGTACTGCTACCCCAGAGTTACCTACCCAGGGAAC
ATTTTCAAATGTTTCTACAAATGTATCCTACCAAGAAACTACAACACCTA
GTACCCTTGGAAGTACCAGCCTGCACCCTGTGTCTCAACATGGCAATGAG
GCCACAACAAACATCACAGAAACGACAGTCAAATTCACATCTACCTCTGT
GATAACCTCAGTTTATGGAAACACAAACTCTTCTGTCCAGTCACAGACCT
CTGTAATCAGCACAGTGTTCACCACCCCAGCCAACGTTTCAACTCCAGAG
ACAACCTTGAAGCCTAGCCTGTCACCTGGAAATGTTTCAGACCTTTCAAC
CACTAGCACTAGCCTTGCAACATCTCCCACTAAACCCTATACATCATCTT
CTCCTATCCTAAGTGACATCAAGGCAGAAATCAAATGTTCAGGCATCAGA
GAAGTGAAATTGACTCAGGGCATCTGCCTGGAGCAAAATAAGACCTCCAG
CTGTGCGGAGTTTAAGAAGGACAGGGGAGAGGGCCTGGCCCGAGTGCTGT
GTGGGGAGGAGCAGGCTGATGCTGATGCTGGGGCCCAGGTATGCTCCCTG
CTCCTTGCCCAGTCTGAGGTGAGGCCTCAGTGTCTACTGCTGGTCTTGGC
CAACAGAACAGAAATTTCCAGCAAACTCCAACTTATGAAAAAGCACCAAT
CTGACCTGAAAAAGCTGGGGATCCTAGATTTCACTGAGCAAGATGTTGCA
AGCCACCAGAGCTATTCCCAAAAGACCCTGATTGCACTGGTCACCTCGGG
AGCCCTGCTGGCTGTCTTGGGCATCACTGGCTATTTCCTGATGAATCGCC
GCAGCTGGAGCCCCACAGGAGAAAGGCTGGAGCTGGAACCCTGA

In one particular embodiment of the invention, the vector used is a lentiviral vector, advantageously the pLenti-III-EF1α lentiviral vector, marketed by the Applied Biological Materials Inc company.

The vector is stably maintained in the human stem cell and may optionally possess particular signals which specify the secretion of the translated protein. These different elements are selected and optimized by the skilled person in the art based on the host cell used. Such vectors are prepared by methods familiar to the skilled person in the art, and the resulting clones may be introduced into a suitable host cell by standard methods, such as lipofection, electroporation, use of polycationic agents, heat shock, or chemical methods.

In another particular embodiment of the invention, the exogenous nucleic acid may be introduced by various methods into chromosome 17, by using, in particular, Transcription Activator-Like Effector Nucleases (TALEN) techniques, grouped into regularly interspaced short palindromic repeats techniques (CRISPR/Cas9), or zinc finger nucleases (ZFN). Advantageously, the integration of exogenous nucleic acid into chromosome 17 is obtained by using regularly interspaced short palindromic repeats (CRISPR/Cas9). The use of this technology in genome editing is well described in the art. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The type II CRISPR is one of the most well characterized systems and carries out targeted DNA double-strand break in four sequential steps. First, two non-coding RNA, the pre-rRNA array and tracrRNA, are transcribed from the CRISPR locus. Second, tracrRNA hybridizes to the repeat regions of the pre-tRNA and mediates the processing of pre-rRNA into mature rRNAs containing individual spacer sequences. Third, the mature tRNA: tracrRNA complex directs Cas9 to the target DNA via Watson-Crick base-pairing between the spacer on the rRNA and the protospacer on the target DNA next to the protospacer adjacent motif (PAM), an additional requirement for target recognition. Finally, Cas9 mediates cleavage of target DNA to create a double-stranded break within the protospacer.

Cas9 is thus the hallmark protein of the type II CRISPR-Cas system, and a large monomeric DNA nuclease guided to a DNA target sequence adjacent to the PAM (protospacer adjacent motif) sequence motif by a complex of two noncoding RNAs: CRISPR RNA (rRNA) and trans-activating rRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. Cas9 expression plasmids for use in the methods of the invention can be constructed as described in the art.

Another object of the invention concerns the genetically modified human stem cell as previously defined for use as a medicinal product. In particular, the present invention relates to the genetically modified human stem cell as defined previously for its use in the prevention and/or treatment of cancer and/or recurrent cancer and/or associated metastases.

Advantageously, the cancer is a cancer selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive.

Advantageously, the invention concerns the genetically modified human stem cell as defined above, for its use in the prevention and/or treatment of solid tumours, in particular hepatocellular carcinomas, and/or recurrent cancer and/or associated metastases.

According to one particularly advantageous embodiment of the invention, the genetically modified human stem cell according to the invention is particularly useful to prevent and/or treat cancer and/or recurrent cancer and/or associated metastases.

According to one particularly advantageous embodiment of the invention, the genetically modified human stem cell according to the invention is particularly useful to prevent and/or treat solid tumours and/or recurrent cancer and/or associated metastases.

According to one particularly advantageous embodiment of the invention, the genetically modified human stem cell according to the invention is particularly useful to prevent and/or treat cancer and/or recurrent cancer and/or associated metastases selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive.

According to one particularly advantageous embodiment of the invention, the genetically modified human stem cell according to the invention is particularly useful to prevent and/or treat hepatocellular carcinomas and/or associated metastases.

Another aspect of the invention concern a method for screening genetically modified stem cells as defined above, characterized in that the method comprises the following steps:
a) transduction of human stem cells with a viral vector comprising the exogenous nucleic acid,
b) culturing of genetically modified human stem cells from step a) in a predefined culture medium, and
c) screening of genetically modified human stem cells expressing the selection marker gene on their membrane surface.

In one particular embodiment of the invention, the culture medium used is a medium specific to human stem cells, such as the medium, Mesenchymal Stem Cell Growth Medium 2, marketed by Promocell (Product Reference: C28009).

Advantageously, the culture medium used comprises 50% of the medium having been in contact with human stem cells not transduced for 24 hrs. and 50% of same fresh medium.

In one particular embodiment, the selection marker gene is the spliced CD34 gene. Advantageously, the screening of genetically modified human stem cells expressing the selection marker gene on their membrane surface is performed by flow cytometry, in particular using the device, BD-FACS ARIAIII™ by using anti-CD34 antibodies coupled to the “brilliant violet 421” fluorochrome (Brilliant Violet 421™ anti-human CD34 antibody (Product Reference: 343609 at Biolegend)).

Another object of the invention concerns a pharmaceutical composition comprising the genetically modified human stem cell according to the invention as an active substance and at least one pharmaceutically acceptable excipient. In one particular embodiment of the invention, the pharmaceutical composition according to the invention comprises a therapeutically effective amount of genetically modified human stem cells as an active substance and at least one pharmaceutically acceptable excipient.

According to one particularly advantageous embodiment of the invention, the pharmaceutical composition comprising a therapeutically effective amount of genetically modified human stem cells as the active substance and at least one pharmaceutically-acceptable excipient as defined above is particularly useful to prevent and/or treat cancer and/or recurrent cancer and/or associated metastases.

Advantageously, the pharmaceutical composition comprising a therapeutically effective amount of genetically modified human stem cells as an active substance and at least one pharmaceutically acceptable excipient as defined above is administered to a patient suffering from or suspected to suffer from cancer and/or relapses of cancer and/or associated metastases.

Advantageously, the cancer is a solid tumour cancer. “Solid tumour” as defined in the current invention refers to a carcinoma or sarcoma. In one particular embodiment of the invention, the cancer is a cancer selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive.

According to one particularly advantageous embodiment of the invention, the pharmaceutical composition comprising a therapeutically effective amount of genetically modified human stem cells as an active substance and at least one pharmaceutically acceptable excipient as defined above is particularly useful to prevent and/or treat solid tumours, and in particular hepatocellular carcinomas, and/or recurrent cancer and/or associated metastases.

Advantageously, the pharmaceutical composition comprising a therapeutically effective amount of genetically modified human stem cells as an active substance and at least one pharmaceutically acceptable excipient as defined above is administered to a patient suffering from or suspected to suffer from solid tumours, and in particular hepatocellular carcinomas, and/or recurrent cancer and/or associated metastases.

In one embodiment, the patient may be a human, male or female, regardless of age. In another embodiment, the patient may be non-human animal, advantageously a non-human mammal, e.g. a dog, cat, mouse, rat, hamster, rabbit, chinchilla, horse, cow, pig, sheep, goat or primate.

As defined in the current invention, the term “therapeutically effective” or “treatment effective amount” or “pharmaceutically effective amount” refers to the amount of genetically modified human stem cells needed to inhibit or reverse a disease condition, advantageously a cancer, and in particular to treat solid tumours and notably hepatocellular carcinomas, and/or recurrent cancer and/or associated metastases. Determining a therapeutically effective amount specifically depends on factors such as toxicity and efficacy of the medicinal product. These factors will differ depending on other factors such as potency, relative bioavailability, patient's body weight, severity of side effects and preferred mode of administration. Toxicity may be determined using methods well-known to in the art. The same is applicable as regards efficacy. A pharmaceutically effective amount, therefore, is an amount that is deemed by the clinician to be toxicologically tolerable, yet efficacious.

Dosage may be adjusted appropriately to achieve desired medicinal product (e.g. genetically modified human stem cells) levels, local or systemic, in particular trans-arterial, depending upon the method of administration. In the event that the patient response is insufficient at such doses, even higher doses (or effective higher doses by a different, more localised delivery route) may be used where the patient tolerance permits. Several injections approximately one week apart may also be used to achieve appropriate levels of toxic metabolites formed to cause the death of tumour cells and triggering an anti-tumour immune response. “Dose” and “dosage” are used interchangeably herein.

The frequency of injections is related to the half-life of the genetically modified human stem cells.

In an advantageous embodiment, the pharmaceutical composition according to the invention is administered to the patient at the rate of one administration per week for at least four weeks. Advantageously, the pharmaceutical composition according to the invention is administered to the patient at the rate of two administrations, advantageously three administrations, advantageously four administrations, advantageously five administrations, advantageously six administrations, advantageously seven administrations per week. Advantageously, the pharmaceutical composition according to the invention is administered for at least four weeks, advantageously at least five weeks, advantageously at least six weeks, advantageously at least seven weeks, advantageously at least eight weeks, advantageously at least nine weeks, advantageously at least ten weeks.

In an advantageous embodiment, the pharmaceutical composition according to the invention further comprises at least one second active substance. In one particularly advantageous embodiment of the invention, the at least one second active substance may interact synergistically with the genetically modified human stem cells according to the invention.

Advantageously, the second active substance is an anti-cancer agent. As an example of a second active substance, mention may be made to cytostatic agents, cytotoxic agents, cytoprotective agents, growth inhibitory agents, toxins and combinations thereof. Examples of anti-cancer agents that may be used in tumour therapy with genetically modified human stem cells of the present invention comprise cyclophosphamide (CAS number 50-18-0, also known as cyclophosphane, and under the trademarks Endoxan®, Neosar®, Procytox® and Revimmune®), AQ4N (1,4)-bis-{[2-(dimethylamino-N-oxide) ethyl]amino} 5,8-dihydroxyanthracene-9,10-dione, also known as Banoxantrone®), ifosfamide (CAS number 3778-73-2), bezyloxyresorufine, 7-Ethoxy-4-trifluoro-methyl-coumarine (CMO), Bupropion, thiotepa (N′N′-triethylenethiophosphoramide, CAS number 52-24-4), mitomycin C (CAS number 50-0-07), tirapazamine (SR-4233, CAS number 27314-97-2). As example of a cytoprotective agent, mention may be made to mesna, also known under the trademark Uromitexian®. Advantageously, the second active substance may comprise a combination of anti-cancer agents, such as those listed above. In one particularly advantageous embodiment, the second active substance is a combination of an anti-cancer agent and a cytoprotective agent. In one particularly advantageous embodiment, the second active substance is a combination of cyclophosphamide and mesna.

In one particular embodiment, the pharmaceutical composition according to the invention comprises genetically modified human stem cells as defined above as the active substance and cyclophosphamide as the second active substance.

In one particular embodiment, the pharmaceutical composition according to the invention comprises genetically modified human stem cells as defined above as the active substance and mesna as the second active substance.

In one particular embodiment according to the invention, the second active substance may be in the form of granules, powders, tablets, film-coated tablets, (micro) capsules, syrups, emulsions, suspensions or extended-release formulations. In another particular embodiment of the invention, the second active substance can be in a form which can be administered parenterally.

In one particular embodiment, the pharmaceutical composition according to the invention comprises genetically modified human stem cells as defined above as the active substance and cyclophosphamide as the second active substance and at least one pharmaceutically acceptable excipient.

In one particular embodiment, the pharmaceutical composition according to the invention comprises genetically modified human stem cells as defined above as the active substance and cyclophosphamide and mesna as the second active substance and at least one pharmaceutically acceptable excipient.

In one advantageous embodiment of the invention, when the pharmaceutical composition comprises a second active substance, the pharmaceutical composition is in a form suitable for simultaneous administration or sequential administration of the genetically modified human stem cells and the second active substance.

As defined in the current invention, “simultaneous administration” refers to the administration of genetically modified human stem cells and the second active substance at the same time, in one and the same administration, to one and the same patient, in therapeutically effective amounts to allow the synergistic effect of the pharmaceutical composition. This does not mean, however, that the genetically modified human stem cells and the second active substance are necessarily administered as a mixture; they may in fact be administered simultaneously but separately, as separate compositions.

“Present in two separate compositions” refers to the fact that the genetically modified human stem cells and the second active substance are physically separate. They are then used, administered separately in amounts that are therapeutically effective in order to allow the synergistic effect of the pharmaceutical composition, without prior mixture, in several (at least two) dosage forms (e.g. two distinct compositions).

In another particular embodiment of the invention, the genetically modified human stem cells and the second active substance may be present in the same composition in therapeutically effective amounts to allow the synergistic effect of the pharmaceutical composition. As defined in the current invention, “present in the same composition” shall be understood to mean the physical combination of genetically modified human stem cells and the second active substance. The genetically modified human stem cells and the second active substance are then administered simultaneously since they are administered together, as a mixture, in one and the same dosage form (e.g. in one composition containing genetically modified human stem cells and the anti-cancer agent) and in therapeutically effective amounts to allow the synergistic effect of the pharmaceutical composition.

As defined in the current invention, “sequential administration” means that the genetically modified human stem cells and the second active substance are administered in therapeutically effective amounts to allow the synergistic effect of the pharmaceutical composition, not simultaneously but separately in time, one following the other. The terms “precede” or “preceding” and “follow” or “following” then apply. The term “precede” or “preceding” is used when a compound of the pharmaceutical composition according to the invention is administered a few minutes or several hours, or even several days prior to the administration of the other compound(s) of the pharmaceutical composition. Conversely, the term “follow” or “following” is used when a compound of the pharmaceutical composition is administered a few minutes or several hours, or even several days after administration of the other compound(s) of the pharmaceutical composition. In one particularly advantageous embodiment of the invention, genetically modified human stem cells are administered a few days prior to the second active substance.

In one advantageous embodiment of the invention, when the pharmaceutical composition comprises a second active substance, the pharmaceutical composition is suitable for the sequential administration of genetically modified human stem cells and the second active substance. Advantageously, the second active agent is cyclophosphamide alone or in combination with mesna.

In one particular embodiment of the invention, the genetically modified human stem cells are first administered to the patient, then, two to three days after the administration of the genetically modified human stem cells, the cyclophosphamide is administered, alone or in combination with mesna, to the same patient, and this at the rate of two administrations of genetically modified human stem cells and cyclophosphamide, alone or in combination with the mesna, per week for at least four weeks, advantageously for at least five weeks, advantageously for at least six weeks, advantageously for at least seven weeks, advantageously for at least eight weeks, advantageously for at least nine weeks, advantageously for at least ten weeks.

Depending on the intended mode of administration in vivo, the pharmaceutical composition used may be in the dosage form of solid, semi-solid or liquid such as e.g., tablets, pills, powders, capsules, gels, ointments, liquids, suspensions or the like. Preferably, the pharmaceutical compositions are administered in unit dosage forms suitable for single administration of precise dosage amounts. The pharmaceutical compositions may also include, depending on the desired formulation, at least one pharmaceutically acceptable carrier or diluent, which are defined as aqueous-based vehicles commonly used in formulations for human administration. The diluent is selected so as not to affect the biological activity in active compounds of the pharmaceutical composition of the invention. Examples of such are diluents are distilled water, physiological saline, Ringer's solution, dextrose solution, and Hank's solution. Effective amounts of such diluent or carrier are amounts which are effective to obtain a pharmaceutically acceptable formulation in terms of solubility of components, biological activity, etc. In some embodiments, the pharmaceutical compositions provided herein are sterile.

Suitable liquid pharmaceutical preparation forms may notably comprised aqueous or saline solutions, said aqueous or saline solutions can be microencapsulated, encochleated, coated onto microscopic gold particles, contained in liposomes.

Administration of the pharmaceutical composition or the genetically modified human stem cell according to the present invention may be by any route, including oral, topical, parenteral (also called systemic or general route), intramuscular, intranasal, sublingual, intratracheal, inhalation, occular, vaginal, and rectal. Intracapsular, intravenous, trans-arterial, intra-arterial, as well as intraperitoneal route of administration may also be used. The skilled person in the art will choose the appropriate route of administration depending on the disorder to be treated.

Advantageously, the pharmaceutical composition or the genetically modified human stem cell according to the present invention can be administered to a subject by the parenteral route, advantageously by trans-arterial route.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulator agents such as suspending, stabilizing and/or dispersing agents.

Pharmaceutical formulations for parenteral administration include aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g. sterile non-pyrogenic water, before use.

In a yet more advantageous manner, the pharmaceutical composition or the genetically modified human stem cell according to the present invention can be administered to a patient by trans-arterial route. According to one particular embodiment of the invention, the pharmaceutical composition or the genetically modified human stem cell according to the invention is in a form suitable for its administration by trans-arterial route.

In one particular advantageous embodiment, the genetically modified human stem cells according to the present invention represent at a rate of 0.5 to 98% in weight, or more, of the total weight of the pharmaceutical composition considered.

In one particularly advantageous embodiment of the present invention, the pharmaceutical composition according to the invention solely comprises the genetically modified human stem cells as defined above, as a unique active substance.

Another object of the invention relates to a method for the preventive treatment of cancer and/or recurrent cancer and/or associated metastases, comprising the administration to the patient of genetically modified human stem cells or a pharmaceutical composition comprising a therapeutically effective amount of genetically modified human stem cells as the active substance and at least one pharmaceutically acceptable excipient as defined above.

As defined in the current invention, the term “prevention” or “prophylaxis” or “preventative treatment” or “prophylactic treatment” comprises a treatment leading to the prevention of a disease as well as treatments reducing and/or delaying the incidence of a disease or the risk of it occurring.

According to the present invention, the genetically modified human stem cell is particularly useful for preventing the recurrence and occurrence of new metastases, provoking a strong anti-tumour immune response, thus inducing a protective effect against recurrence and metastases.

In one particularly advantageous embodiment, the present invention relates to a method for the preventive treatment of cancer and/or recurrent cancer and/or associated metastases, comprising the administration to the patient of genetically modified human stem cells or a pharmaceutical composition comprising a therapeutically effective amount of genetically modified human stem cells as the active substance and at least one pharmaceutically acceptable excipient as defined above and a second active substance as also defined above.

Advantageously, the cancer is a solid tumour cancer. “Solid tumour” as defined in the current invention refers to a carcinoma or sarcoma. In one particular embodiment of this the invention, the cancer is a cancer selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive.

Advantageously, the cancer is a solid tumour, in particular a hepatocellular carcinoma, and/or recurrent cancer and/or associated metastases.

In one particularly advantageous embodiment, the present invention relates to a method for the preventive treatment of hepatocellular carcinoma, comprising administration to the patient of genetically modified human stem cells or a pharmaceutical composition comprising a therapeutically effective amount of genetically modified human stem cells as an active substance and at least one pharmaceutically acceptable excipient as defined above.

In one particularly advantageous embodiment, the present invention relates to a method for the preventive treatment of hepatocellular carcinoma, comprising administration to the patient of genetically modified human stem cells or a pharmaceutical composition comprising a therapeutically effective amount of genetically modified human stem cells as an active substance and at least one pharmaceutically acceptable excipient as defined above and a second active substance as defined above.

In one particularly advantageous embodiment, the present invention relates to the use of a pharmaceutical composition comprising a therapeutically effective amount of genetically modified human stem cells as an active substance in the manufacture of a medicinal product intended for the prevention of cancer and/or recurrent cancer and/or associated metastases.

Advantageously, the cancer is a solid tumour cancer. “Solid tumour” as defined in the current invention refers to a carcinoma or sarcoma. In one particular embodiment of this the invention, the cancer is a cancer selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive Advantageously, the cancer is a solid tumour, in particular hepatocellular carcinoma and/or associated metastases.

In one particularly advantageous embodiment, the present invention relates to the use of a pharmaceutical composition comprising a therapeutically effective amount of genetically modified human stem cells as an active substance for the manufacture of a medicinal product intended for the prevention of solid tumours, and in particular hepatocellular carcinomas, and/or recurrent cancer and/or associated metastases.

Another object of the invention relates to a method for the curative treatment of cancer and/or recurrent cancer and/or associated metastases in patients in need of an administration, comprising the administration to such patients of genetically modified human stem cells or a pharmaceutical composition comprising a therapeutically effective amount of genetically modified human stem cells as the active substance and at least one pharmaceutically acceptable excipient as defined above.

As used herein, the term “treatment” or “curative treatment” is defined as a treatment leading to a cure or a treatment which alleviates, improves and/or eliminates, reduces and/or stabilizes the symptoms of a disease or the suffering that it causes.

Another object of the invention relates to a method for the curative treatment of cancer and/or recurrent cancer and/or associated metastases in patients in need of an administration, comprising the administration to such patients of genetically modified human stem cells or pharmaceutical compositions comprising a therapeutically effective amount of genetically modified human stem cells as the active substance and at least one pharmaceutically acceptable excipients as defined above and a second active substance as defined above.

Advantageously, the cancer is a solid tumour cancer. “Solid tumour” as defined in the current invention refers to a carcinoma or sarcoma. In one particular embodiment of this the invention, the cancer is a cancer selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive. Advantageously, the cancer is a solid tumour, in particular a hepatocellular carcinoma, and/or recurrent cancer and/or associated metastases.

Another object of the invention relates to a method for curative treatment of a solid tumour, advantageously hepatocellular carcinomas, and/or recurrent cancer and/or associated metastases, in patients in need of an administration, comprising the administration to such patients of genetically modified human stem cells, or a pharmaceutical composition comprising a therapeutically effective amount of genetically modified human stem cells as the active substance and at least one pharmaceutically acceptable excipient as defined above.

Another object of the invention relates to a method for curative treatment of a solid tumour, advantageously hepatocellular carcinomas, and/or recurrent cancer and/or associated metastases, in patients in need of an administration, comprising the administration to such patients of genetically modified human stem cells, or a pharmaceutical composition comprising a therapeutically effective amount of genetically modified human stem cells as the active substance and at least one pharmaceutically acceptable excipient as defined above and a second active substance as defined above. In one particularly advantageous embodiment, the present invention relates to the use of a pharmaceutical composition comprising a therapeutically effective amount of genetically modified human stem cells as the active substance in the manufacture of a medicinal product intended for the curative treatment of cancer and/or recurrent cancer and/or associated metastases.

Advantageously, the cancer is a solid tumour cancer. “Solid tumour” as defined in the current invention refers to a carcinoma or sarcoma. In one particular embodiment of this the invention, the cancer is a cancer selected from: cancers affecting the central nervous system, such as astrocytomas and gliomas; upper respiratory tract cancers (ENT), such as cancer of the lips, cancer of oral cavity, cancer of the oropharynx and cancer of the nasopharynx; cancers of the endocrine glands, such as thyroid cancer, adrenal cancer, and extra-digestive and pulmonary neuroendocrine tumors; cancers of the exocrine glands, such as breast cancer and pancreatic cancer; cancers affecting the chest, such as cancer of the pleura and lung cancer; digestive cancers, such as cancer of the oesophagus, cancer of the stomach, cancer of the small intestine, cancer of the colon, and cancer of the rectum and/or anal canal; genital cancers, such as prostate cancer, cervical cancer, endometrial cancer, cancer of the vagina and cancer of the vulva; urinary cancers, such as kidney cancer and bladder cancer; sarcomas, such as soft tissue sarcoma, gastrointestinal stromal tumors (GIST), and bone sarcoma; skin cancers, such as melanoma, hepatobiliary cancers, such as hepatocellular carcinoma and cholangiocarcinoma, the list is not exhaustive.

In one particularly advantageous embodiment, the present invention relates to the use of a pharmaceutical composition comprising a therapeutically effective amount of genetically modified human stem cells as the active substance in the manufacture of a medicinal product intended for the curative treatment of solid tumours, and in particular hepatocellular carcinomas, and/or recurrent cancer and/or associated metastases.

### FIGURES

FIG. 1 is a schematic representation of the nucleic acid comprising the vectorized nucleic acid (SEQ ID NO. 8).

FIG. 2 represents the determination of IC50 in TC1 cells, after the transfer of supernatants of human mesenchymal stem cells (hMSC), human mesenchymal stem cells having integrated the gene encoding for the fusion protein (hMSC*), clones 3, 4 and 10, said cells having been treated previously for 24 hrs. with increasing concentrations of cyclophosphamide (CPA).

FIG. 3 represents the exposure of calreticulin (CRT) on the surface of TC1 cells treated for 24 hrs. with supernatants of human mesenchymal stem cells (hMSC), TC1 cells having integrated the gene encoding for the fusion protein (TC1 CYP2B6*), genetically modified human stem cells according to the invention (hMSC-CYP2B6*) and murine mesenchymal stem cell (mMSC) having integrated the gene encoding for the fusion protein (mMSC-CYP2B6*), said cells having been treated previously for 24 hrs. with a dose of 250 μM of cyclophosphamide (CPA).

FIG. 4 represents the FACS quantification of the dying TC1 population treated for 48 hrs. using supernatants of human mesenchymal stem cells (hMSC), the TC1 cells having integrated the gene encoding for the fusion protein (TC1 CYP2B6*), genetically modified human stem cells according to the invention (hMSC-CYP2B6*) and murine mesenchymal stem cell having integrated the gene encoding for the fusion protein (mMSC-CYP2B6*), said cells having been treated previously for 24 hrs. with a dose of 250 μM cyclophosphamide (CPA).

FIG. 5 represents the ELISA quantification of the HMGB1 concentration released by dying TC1 cells treated for 48 hrs. with supernatants of human mesenchymal stem cells (hMSC), TC1 cells having integrated the gene encoding for the fusion protein (TC1 CYP2B6*), genetically modified human stem cells according to the invention (hMSC-CYP2B6*) and murine mesenchymal stem cells having integrated the gene encoding for the fusion protein (mMSC-CYP2B6*), these cells having been treated previously for 24 hrs. with a dose of 250 μM cyclophosphamide (CPA).

FIG. 6 represents the increase of the ratio of genetically modified human stem cells according to the invention (hMSC-CYP2B6*) treated with 250 μM of cyclophosphamide (CPA) on six different cell lines, Huh7, SNU398, SNU 387, SNU 878, MHCC97H and PLC/PRF/5.

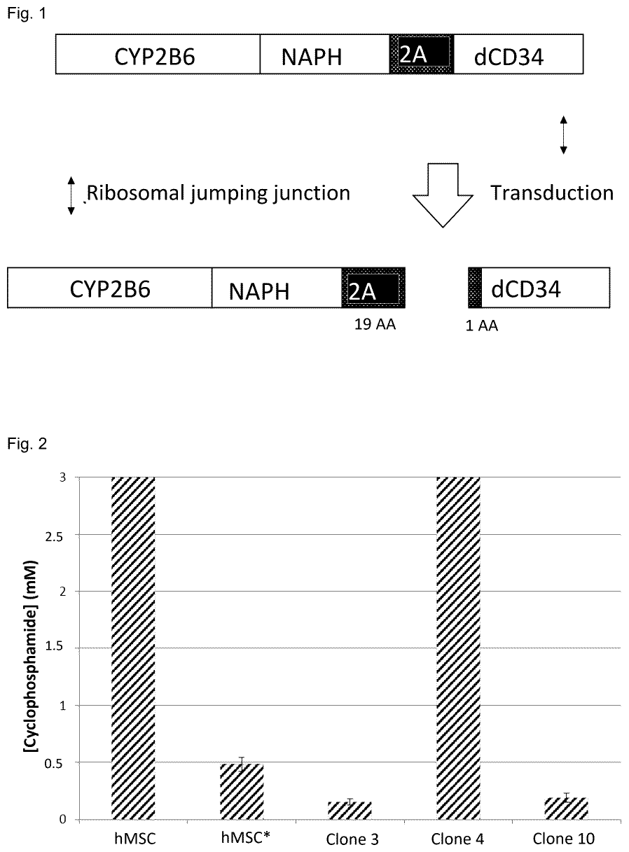

### EXAMPLES

### Example 1: Method for Obtaining the Human Stem Cells in the Invention

The cells used are human mesenchymal stem cells (hMSC) sold by PromoCell, obtained from fatty tissues and cultured in a special medium sold by the manufacturer, the Mesenchymal Stem Cell Growth Medium 2, containing only 2% of Foetal Bovine Serum (FBS). On D−1 of the transduction, the hMSC are detached using accutase (StemPro® Accutase®, GIBCO), which is a less stringent product than trypsin and inoculated at 7.5·10⁴ cells in different wells of a 6-well plate (9.6 cm²).

On day D, the cells are transduced with 50 lentiviral particles expressing the exogenous nucleic acid encoding the fusion protein per cell (50 MOI). Dilution of the lentiviral particles is done in a volume of culture medium, PromoCell Growth Medium 2, of 800 μL per well. The cells are then incubated at 37° C. After 4 hours, 1.2 mL of the medium (PromoCell Growth Medium 2) is added to each well. The transduction is stopped at D+1, by removing the medium containing the lentiviruses and by rinsing with PBS. Then, 2 mL of the medium are added to each well. Depending on the growth rate of transduced cells, these are collected and transferred in T75 (75 cm² flask) on D+2 or D+3. Once amplified, the cells are then marked with the anti-CD34 antibody, and sorted by using FACS.

### Example 2: Determination of the Number of Copies of the Exogenous Nucleic Acid Encoding the Fusion Protein Integrated in the Human Stem Cells in the Invention

The transduced cells have a CD34 marking on the surface that is more or less visible allowing the discrimination of the cells containing the exogenous nucleic acid encoding the fusion protein, or not. The dead cells are visible thanks to propidium iodide (PI) and are excluded. A pool of transduced cells has been created from a minimum fluorescence of 2×103 “Arbitrary Unit” (AU) of CD34 corresponding to approximately 5% of total cells while a more stringent screening starting from 104 AU of CD34, corresponding to approximately 3% of cells, was made in order to obtain a clone by inoculated these cells at a rate of one cell per well (plate of 96 wells).

From a pool of transduced cells, 5 plates of 96 wells (P96P) were inoculated in order to isolate clones (1 cell per well), and by cultivating these, by using for three plates of the five plate the hMSC medium and for two plates of the five plates the medium conditioned by the hMSC. This conditioned medium is a 1/1 mix of the fresh medium and a medium that has been in contact with proliferating hMSC.

Parallel to this verification, a cytotoxic test using the 3-(4,5 dimethylthiazol-2 yl)-5-(3-carboxymethoxyphenyl)-2-(4-sulfophenyl)-2H-tetrazolium (MTS) was performed. This test aims to measure the cell viability in the presence of CPA. The MTS is reduced by the mitochondria of viable cells in a soluble and stained product, formazan. The absorbance measure at 490 nm is an estimate of the cytotoxic effect of CPA, compared to a control well.

The pool and each of the clones were incubated for 72 hrs. with increasing doses of CPA in order to identify sensitive clones. This test has shown that the pool was sensitive to CPA (IC50=2 mM), and allows to identify 2 clones sensitive to CPA (clone 3, IC50=300 μM; clone 10, IC50=450 M) while all the others were not. We checked beforehand that the naïve hMSC were not sensitive to CPA.

Another clone, clone 4, was selected (IC50>3 mM) in order to understand why the cells were expressing CD34 on their surface but were not sensitive to CPA. Using Polymerase Chain Reaction (digital PCR), we analysed the hMSC, hMSC* (hMSC having integrated the gene encoding for the fusion protein), clone 3 (C3) clone 4 (C4) and clone 10 (C10) in order to determine the number of copies of the inserted gene. Indeed, the digital PCR was optimised in order to detect circulating tumoral DNA. The principle here is based on the generation of a multitude of drops containing a maximum DNA molecule based on a Poisson distribution. Each drop containing DNA will then undergo a PCR, with 2 couples primers and 2 Taqman™ probes. The first probe will recognize an internal reference, in this case the presence of ribonuclease P (or “RNase P”) in humans, coupled with the fluorochrome VIC® “2′-chloro-7′-phenyl-1,4-dichloro-6-carboxyfluorescein”. The second probe, specifically designated for our gene suicide, is coupled with fluorochrome FAM™ “6-carboxyfluorescein”. These 2 fluorochromes will be inhibited in their probes by the (tetramethylrhodamine) TAMRA™ quencher. Thus, the outcomes of the experiment can be categorized according to 3 possibilities:
A drop without DNA, or with a DNA that has no target sequence, shall be a drop considered blank; or
A drop containing a target sequence will be detected as FAM positive or VIC positive; or
A drop containing the 2 target sequences will be detected as FAM and VIC positive with a colour mixture of the 2 target sequences.

The VIC positive drops will determine the number of copies of the internal reference of the sample making it possible to transform a theoretical DNA amount into an actual number of DNA molecules. The report of FAM positive-drops on the VIC positive drops will then make it possible to determine the number of copies by multiplying it by 2. In fact, the internal reference containing 2 copies of the gene per genome.

This analysis enabled us to detect a single copy of our transgene in the three different clones C3, C4 and C10 but also in the pooled transduced cells.

In the aim of testing these clones, new MTS tests, where, this time, the human clones are treated for 24 hrs. with increasing concentrations of CPA were performed. The supernatant is then collected and transferred onto TC1 cells. The human mesenchymal stem cells (hMSC) and the clone 4 possesses a IC50>3 mM CPA, the maximum dose tested, whilst the IC50 of approximately 500, 150 and 200 μM corresponds to hMSC*, to clone 3 and to clone 10 respectively (see FIG. 2).

### Example 3: Determination of Insertion Site of the Exogenous Nucleic Acid Encoding for the Fusion Protein

The clones 3, 4 and 10 were sequenced in order to discover the exact insertion site of the exogenous nucleic acid encoding for the fusion protein in these clones.

This insertion occurred in chromosome 11 for clone 4 but with the deletion of 490 nucleotides in the middle of the exogenous nucleic acid, explaining as such why this clone was not sensitive to CPA. In addition to an IC50 slightly superior to clone 3, clone 10 demonstrated a fragility to proliferate over time and a morphologic evolution of the cells. Sequencing revealed that the insertion of exogenous nucleic acid occurs in chromosome 6 of clone 10. This area of insertion is subject to many chromosomal rearrangements surely explaining the difficulty of cells to proliferate over time. Clone 3 possesses an insertion of exogenous nucleic acid encoding the fusion protein in the gene ZZEF1 (“Zinc Finger ZZ-Type And EF-Hand Domain Containing 1”) of chromosome 17.

Following these results, clone 3 was selected to continue the tests in vitro. We then compared the human mesenchymal stem cells (hMSC), the genetically modified human stem cells according to the invention (hMSC-CYP2B6*) (clone 3), murine mesenchymal stem cells (mMSC) having integrated the gene i encoding for the fusion protein (mMSC-CYP2B6*) and tumour cells TC1 having integrated the gene encoding for the fusion protein (TC1-CYP2B6*). The presence of a copy of the exogenous nucleic acid encoding the fusion protein has been verified by digital PCR, within the TC1-CYP2B6* and a ratio of IC₅₀ has been established to have the most correct possible comparison.

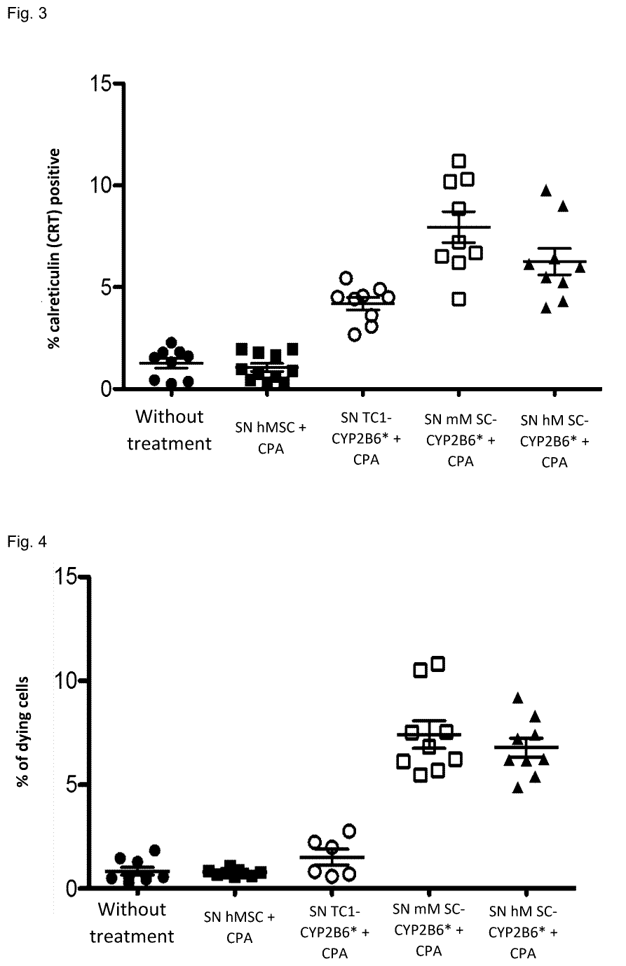

On the basis of ICD, 3 molecular profiles associated with major damage (DAMPs or Damage Associated Molecular Pattern) were tested. Exposure to Calreticulin (CRT) was measured by FACS 24 hrs. after transfer on TC1 of the supernatant of mesenchymal stem cells previously treated for 24 hrs. with 250 μM CPA. The supernatant of mesenchymal stem human cells or of murine mesenchymal stem cells treated with CPA induce the translocation of CRT to the surface of tumoral cells (see FIG. 3).

The TC1 cells were then in contact for 48 hrs. with supernatant of cells previously treated for 24 hrs. with a dose of 250 μM CPA. The TC1 cells were collected and marked with quinacrine in order to permit visualization of intracellular ATP. The “dying cells” possess intracellular ATP levels that are very low and this decrease corresponds to the sustained release of ATP occurring during the immunogenic death of cells (ICD or Immunogenic cell death). The different populations of dying cells found are presented in FIG. 4.

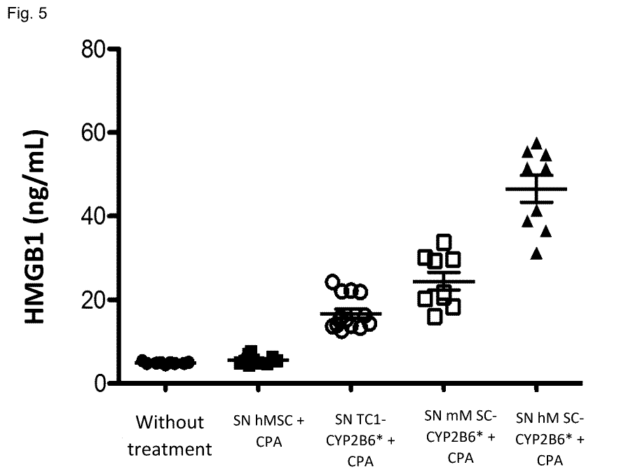

In parallel, the supernatant is collected and tested using an ELISA test to detect the extracellular amount of HMGB1 (high mobility group box 1). FIG. 5 collects the results of the different concentrations of HMGB1 in the tested supernatant.

The TC1-CYP2B6* showed a poorer cytotoxic effect on TC1 than the hMSC-CYP2B6* or mMSC-CYP2B6*. However, more importantly, the TC1-CYP2B6* demonstrate a lower efficacy in triggering the immunogenic death of tumor cells, a key point of our strategy. If, at the level of the CRT translocation, the treatment with the supernatant (SN) of TC1-CYP2B6* is only slightly but significantly (p<0.05) lower than the supernatants of mMSC-CYP2B6* and hMSC-CYP2B6*, the number of dying cells containing small amounts of ATP is significantly lower (p<0.001).

Indeed, the 3 major DAMPs of the ICD, are less well detected in the TC1, after incubation with the supernatant of TC1-CYP2B6* than with the supernatant of hMSC-CYP2B6* or mMSC-CYP2B6* having the gene. These results show that more than a vectorization, mesenchymal stem cells provide a real direct cytotoxic advantage and potentiate the triggering of the immune system.

### Example 4: Evaluation of the Cytotoxic Effect of Human Stem Cells According to the Invention on Hepatocellular Carcinoma Cells

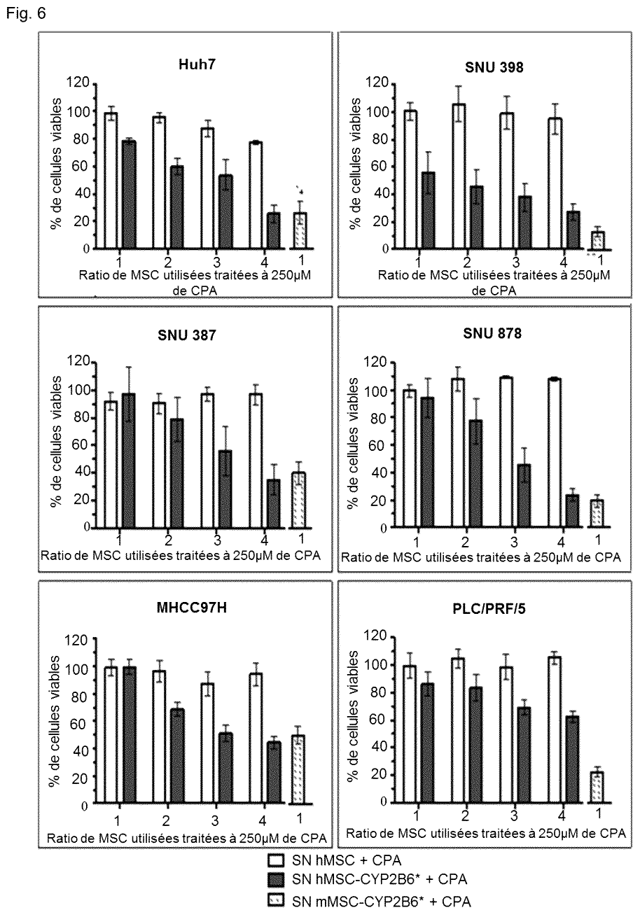

Cytotoxic tests using 3-(4.5 dimethylthiazol-2-yl)-5-(3-carboxymethoxyphenyl)-2-(4-sulfophenyl)-2H-tetrazolium (MTS) were performed on the supernatant of hMSC, the hMSC*, the mMSC and mMSC-CYP2B6* obtained from 6 different cell lines (Huh7, SNU398, SNU 387, SNU 878, MHCC97H and PLC/PRF/5). All of the IC50 of the mMSC and hMSC without the gene encoding for the fusion protein were found to be greater than 3 mM of cyclophosphamide (CPA) for the 6 cell lines. The different IC₅₀ of the mesenchymal stem cells having integrated the gene encoding for the fusion protein (mMSC-CYP2B6*) and genetically modified human stem cells according to the invention (hMSC-CYP2B6*) for the cell lines are listed in FIG. 6. All of the cell lines are sensitive to the supernatant of mMSC-CYP2B6* while the clone hMSC-CYP2B6* appears to have difficulty to trigger cytotoxic effects on the SNU387 and PLC/PRF/5 lines. For each cell line, the mMSC-CYP2B6* demonstrate an IC₅₀ lower than the hMSC-CYP2B6*. This systematic difference can be explained by the number of copies integrated in the target genome. Indeed, unlike hMSC-CYP2B6* which possess only one copy of the gene, the mMSC-CYP2B6* contain 4 copies.

To offset this deviation, we increased the number of genetically modified human stem cells according to the invention (hMSC-CYP2B6*) compared with tumor cells in order to artificially increase the number of copies even if this logic does not allow for a perfect comparison. Indeed, a greater number of CYP2B6* inside of a single cell does not correspond to the same number of CYP2B6* dispersed in multiple cells. An even greater number of treated cells may also influence the cytotoxic effect.

However, we performed new MTS tests using a fixed dose of cyclophosphamide (CPA) at 250 μM with increasing amount of cells (1, 2, 3 and 4 times more cells than mMSC-CYP2B6*).

This new test highlights that the supernatants of cells according to the invention (hMSC-CYP2B6*) caused a cytotoxic effects on all cell lines. The increasing amount of cells according to the invention (hMSC-CYP2B6*) leads to an increasingly powerful effect. The 4-fold increase makes it possible to achieve approximately the same level as the mMSC-CYP2B6*.

1. An isolated genetically modified human stem cell, wherein the human stem cell comprises an exogenous nucleic acid comprising a region encoding a fusion protein comprising a mutant human cytochrome P450 2B6 protein (CYP2B6*) of SEQ ID No. 1 or a variant or fragment thereof, said variant or fragment having at least 80% identity with the amino acid sequence SEQ ID No. 1 and comprising the residues 114V, 199M and 477W as shown in the amino acid sequence SEQ ID No. 1; and a NADPH-cytochrome P450 reductase protein of SEQ ID No. 2 or a variant or fragment thereof, said variant or fragment having at least 80% identity with the amino acid sequence SEQ ID No. 2, operably linked to a promoter, said exogenous nucleic acid having been inserted into the intron located between exon 3 and exon 4 of the ZZEF1 gene at site 4115336 on chromosome 17 of said human stem cell.

2. The isolated genetically modified human stem cell according to claim 1, wherein said human stem cell is chosen from among mesenchymal stem cells (MSC), induced pluripotent stem cells (iPSC) and induced mesenchymal stem cells (iMSC).

3. The isolated genetically modified human stem cell according to claim 1, wherein the promoter is a constitutive promoter.

4. The isolated genetically modified human stem cell according to claim 1, wherein the exogenous nucleic acid further comprises a selection marker gene.

5. The genetically modified human stem cell according to claim 1, wherein the human stem cell was obtained by retroviral transduction with a viral vector comprising the exogenous nucleic acid.

6. A method for obtaining and screening genetically modified stem cells according to claim 1, wherein the method comprises the steps of:
a) transduction of human stem cells with a viral vector comprising the exogenous nucleic acid, said exogenous nucleic acid comprising a selection marker gene,
b) culturing the genetically modified human stem cells of step a) in a predefined culture medium,
c) screening the genetically modified human stem cells expressing the selection marker gene on their membrane surface.

7. A pharmaceutical composition comprising the genetically modified human stem cell according to claim 1 as an active substance and at least one pharmaceutically acceptable excipient.

8. The pharmaceutical composition according to claim 7, further comprising at least one second active substance.

9. The pharmaceutical composition according to claim 8, wherein the second active substance is an anti-cancer agent.

10. The pharmaceutical composition according to claim 7 characterized in that it is in a suitable form for its administration by trans-arterial route.

11. A method for the prevention and/or treatment of cancer and/or associated metastases comprising administering the pharmaceutical composition of claim 7 to a patient.

12. The method of claim 11, wherein the cancer is a solid tumour.

13. The method of claim 11, wherein the cancer is a hepatocellular carcinoma.

14. The genetically modified human stem cell of claim 1, wherein the genetically modified human stem cell is not an human embryonic stem cell.

15. The genetically modified human stem cell of claim 3, wherein the promoter is an EF1-α promoter.
